(19) United States
(12) Patent Application Publication
(10) Pub. No.: US 2008/0153788 A1
(43) Pub. Date: Jun. 26, 2008

(54) NOVEL COMPOSITION TO INCREASE MUSCLE STRENGTH

(75) Inventor: Mike FARBER, Montreal (CA)

Correspondence Address: BERESKIN AND PARR, 40 KING STREET WEST, BOX 401, TORONTO, ON, M5H 3Y2, omitted

(73) Assignee: ORAL DELIVERY TECHNOLOGIES, Montreal (CA)

(21) Appl. No.: 11/961,571

(22) Filed: Dec. 20, 2007

Related U.S. Application Data
(60) Provisional application No. 60/871,511, filed on Dec. 22, 2006.

(51) Int. Cl. A61K 31/56; A61P 21/06

(52) U.S. Cl. 514/170

(57) ABSTRACT

The present invention relates to a composition comprising a sufficient amount of at least two ketosteroid derivatives of testosterone's metabolism in association with a pharmaceutically acceptable carrier for increasing testosterone's physiological levels in a male subject.

### BACKGROUND OF THE INVENTION

(a) Field of the Invention

The present invention relates to a composition comprising an effective amount of at least two ketostreoid derivatives of testosterone metabolism in association with a pharmaceutically acceptable carrier for increasing testosterone's physiological levels in a male subject, wherein the increase in testosterone's levels increases libido, muscle strength and/or athletic performance.

(b) Description of Prior Art

Testosterone is the principal male androgen and is responsible for the development and maintenance of male sexual characteristics, including external virilization, sexual maturity at puberty, spermatogenesis, sexual behavior/libido and erectile functioning. It also supports bone and muscle tissue development during growth. However, after physical maturity, the level of circulating testosterone starts to decline, possibly leading to a diminution in muscle mass. Therefore, there is a growing need for the development of some form of androgen replacement, for the treatment of the various side-effects associated with this condition, such as the decrease in libido, sexual functioning and overall sense of being later in life, but most precisely for the elimination of the effects encountered on muscle mass, which is a specific concern for men evolving in the sports and bodybuilding domains.

Several methods for re-establishing androgens levels to their pre-adult concentrations in men were developed with injectable preparations. U.S. Pat. No. 6,989,378, between others, relates to a novel androgen, (7α,17β)-7-methyl-17-[(1-α-oundecyl)oxy]estr-4en-3one (MENT undecanoate), having a good solubility in oily media and being particularly suitable for administration by means of injection. Injectable medias are normally fashioned in order to allow slow and sustained hormone release in the blood of the patient over various preset periods of time. However, the main problem with such inventions is that it usually ends up providing inconsistent dosing because of a great variance in hormone release between the site of injection and the rest of the body. Moreover, injection of testosterone preparations usually entails very high concentrations from the moment of the administration followed by a period of subnormal hormone concentrations. Because of such uncontrolled release of the active agent, various side-effects developed in periods of high hormone concentrations, such as gynecomastia, water retention, edema and increased fat deposition.

Some methods of treatment for restoring the testosterone concentrations in adult males with declining levels focused on the administration of a metabolic precursor of testosterone. U.S. Pat. No. 5,880,117 relates to a method of administering the testosterone precursor 4-androstene-3,17β-diol as a means of increasing testosterone levels in humans. The invention proposes a compound which concentration peaks within 90 minutes of its administration and declines thereafter over a period of 3 to 4 hours. Even if the androgen preparation has shown easy conversion to testosterone in the physiologic environment, it still lacks constant repartition in the organs of predilection and can possibly entail various side-effects. U.S. Pat. No. 6,451,782 is based on the administration of 4-androstene-3α,17β-diol, a direct precursor hormone to testosterone, in order to increase testosterone levels in male subjects. However, even if conversion to testosterone has been demonstrated as being much more complete than in other cases, the release kinetic of the compound were still not ideal.

Other proposed methods of treating the present condition were related to the physiologic administration of synthetic androgen derivatives of testosterone such as methyltestosterone, fluoxymesterone and stanozol. Those compounds were alkylated at the 17^{th} carbon in order to restrain any reduction of the metabolite to its inactive from. Such innovation induced an increase in the bioavailability of the compound, which allowed a more constant release of the active agent in the physiologic environment. However, patients encountered possible risks of developing complications with liver functions, which highly diminishes the convenience of using such technology.

Steroidal based aromatase inhibitors, Androsta-1,4,6-triene-3,17-dione (ATD) specifically, have been cited in the literature on numerous occasions over the past thirty years. It was first used to elucidate the nature of the enzyme aromatizing androstenedione and testosterone. The effects of aromatase inhibition upon sex steroids in men (in this case older eugonadal men) were definitively and quantatively studied wherein it was shown that administration of an aromatase inhibitor to 15 men over 65 caused significant decreases in estrogen and its related compounds and significantly increased testosterone.

It would therefore be highly desirable to be provided with a potent fast acting aromatase inhibitor displaying only slight or negligible binding to the peripheral androgen receptors that would rapidly raise testosterone levels in male subjects, have a half life allowing for at most twice daily ingestion and have sufficient binding to the hypothalamic androgen receptor sites to down-regulate the feedback loop.

### SUMMARY OF THE INVENTION

In accordance with the present invention, there is provided a composition comprising a sufficient amount of at least two ketosteroid derivatives of testosterone's metabolism for increasing testosterone's physiological levels in a male subject in association with a pharmaceutically acceptable carrier, wherein said increase in testosterone's levels increases libido, muscle strength and/or athletic performances.

In accordance with the present invention, the composition comprises four ketosteroid derivatives of testosterone's metabolism.

In accordance with a preferred embodiment of the present invention, the ketosteroid derivative of testosterone's metabolism are chosen from 3,17-diketoandrost-1,4,6-triene, 6-methylene-3-keto-17hydroxylandrost-1,4-diene, 6 bromo-alpha-3,27-diketoandrost-1,4-diene and 6 bromo-beta-3,27-diketoandrost-1,4-diene.

In accordance with a preferred embodiment of the present invention, the four ketosteroid derivative of testosterone's metabolism comprised in the composition are 3,17-diketoandrost-1,4,6-triene, 6-methylene-3-keto-17hydroxylandrost-1,4-diene, 6 bromo-alpha-3,27-diketoandrost-1,4-diene and 6 bromo-beta-3,27-diketoandrost-1,4-diene.

In accordance with a preferred embodiment of the present invention, the pharmaceutically acceptable carrier is a liposomal carrier bound to a saliva-absorbing carrier.

In accordance with still a preferred embodiment of the present invention, the saliva-absorbing carrier is microcrystalline cellulose.

In accordance with the present invention, there is provided a method for improving a male's libido, muscle strength, and/or athletic performances, which comprises administering a sufficient amount of the composition.

In accordance with a preferred embodiment of the present invention, the administration of the composition is peroral.

In accordance with a preferred embodiment of the present invention, the administration of the composition is transdermal, intranasal and sublingual.

In accordance with a preferred embodiment of the present invention, the daily total dosage of the composition to administer is of about 25 to 1000 mg.

In accordance with a preferred embodiment of the present invention, the administration of the total daily dosage is divided at least in two equal dosages to be administered at intervals of twelve hours.

In accordance with still a preferred embodiment of the present invention, testosterone levels are increased to supraphysiological levels.

In accordance with still a preferred embodiment of the present invention, muscle strength and size are dramatically and rapidly increased in response to exercise.

All references referred herein are hereby incorporated by reference.

### BRIEF DESCRIPTION OF THE DRAWINGS

FIG. 1 consists of the molecular structure of 3,17-diketoandrost-1,4,6-triene compound.

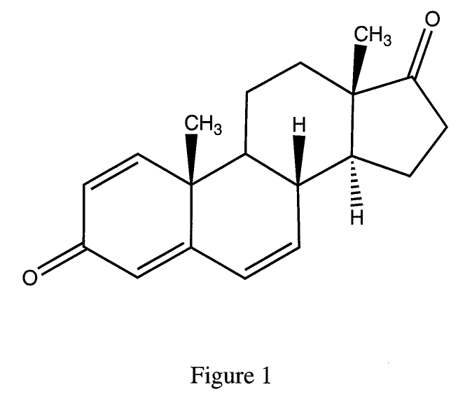

FIG. 2 consists of the molecular structure of the 6-methylene-3-keto-androst-1,4-diene-17-ol.

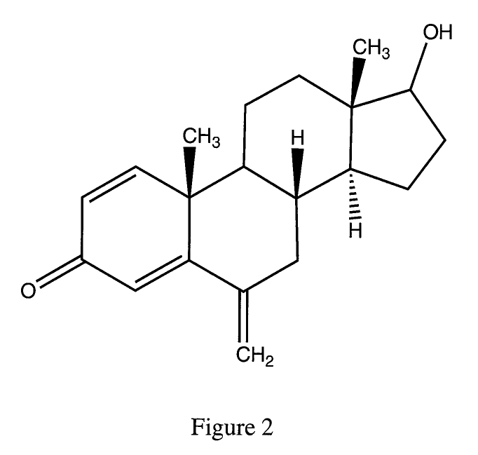

### DETAILED DESCRIPTION OF THE INVENTION

In accordance with the present invention, there is provided a composition comprising a sufficient amount of at least two ketosteroid derivatives of testosterone's metabolism in association with a pharmaceutically acceptable carrier for increasing testosterone's physiological levels in a male subject, wherein the increase in testosterone's levels increases libido, muscle strength and/or athletic performance.

The increase in testosterone's levels in the present invention is generated through administration of preparations of testosterone's ketostreroid metabolic derivatives. Those derivatives are the products of the ketosteroid reduction of this androgen by members of an enzymatic family present in high quantities in steroid target tissues and catalyzing many key reactions: the 3α-hydroxysteroid dehydrogenase family. These enzymes are at the center of various reactions of activation and deactivation of male and female sex hormones, therefore protecting the tissue against circulating hormone excess. The enzymes that are involved in those reactions are the members of the aldo-keto reductase family, or human 3α-hydroxysteroid dehydrogenase isoforms. In humans, this family holds four different 3α-HSD isoforms: type 1 3α-HSD (AKR1C4) [17,1], type 2 3α(17β)—HSD (AKR1C3), type 3 3α-HSD (AKR1C2) and 20α(3α)-HSD (AKR1C1). Type 2 3-α-hydroxysteroid dehydrogenase (3α-HSD) is a multi-functional enzyme that possesses 3α-, 17β- and 20α-HSD, as well as prostaglandin (PG) F synthase activities and catalyzes androgen, estrogen, progestin and PG metabolism. Type 2 3α-HSD was cloned from human prostate, is a member of the aldo-keto reductase (AKR) superfamily and was named AKR1C3. In androgen target tissues such as the prostate, AKR1C3 catalyzes the conversion of Δ-(4)-androstene-3,17-dione to the active testosterone metabolite, testosterone to its inactive C-17-ketosteroids reduced form, the very potent 5α-dihydrotestosterone to the 5α-androstane-3α,17β-diol (3α-diol) inactive metabolite, and 3α-diol to androsterone. Thus, AKR1C3 regulates the balance of androgens and hence trans-activation of the androgen receptor in the prostate, by modulating the concentration of circulating steroid hormone, and therefore generate the growth of the gland and related muscle size and strength increase. Indeed, tissue distribution studies indicate that AKR1C3 transcripts are highly expressed in human prostate.

The main mechanism of action utilized through this invention consists in competitively inhibiting the metabolism of the AKR1C3 isoform of the 3α-hydroxysteroid dehydrogenase family. Indeed, since the metabolic reactions catalyzed by this enzyme is reversible, by introducing testosterone's AKR1C3 metabolic derivative, which is 3,17-diketoandrost-1,4,6-triene, the enzyme will bind to the newly introduced substrate rather than to the circulating testosterone. Thus, rather than degrading testosterone, AKR1C3 will actually perform an oxidative metabolism on the presented 3,17-diketoandrost-1,4,6-triene molecule that will result in the production of testosterone. Therefore, by administrating testosterone's metabolic derivatives in the milieu, the enzyme is inhibited in its ketosteroid reduction of testosterone in inactive metabolites, and will instead perform the inverse reaction, consisting in transforming testosterone's inactive metabolites in testosterone potent molecules. Thus, testosterone's levels are increased by metabolism of the androgen from its inactive metabolite and related inhibition of the metabolism of the physiologic circulating hormone.

The composition has sufficient amounts of ATD to interfere with the circulating aromatase enzymes over a period of twelve hours after initial inhibition by the 6 bromo-α-3,27-diketoandrost-1,4-diene and 6 bromo-β-3,27-diketoandrost-1,4-diene compounds but not enough ATD to have antagonistic competition for the peripheral androgens. Therefore, the ratios of the various compounds in the composition are determined by the need for rapid and effective downregulation of the aromatase enzymes by the 6 bromo-α-3,27-diketoandrost-1,4-diene and 6 bromo-β-3,27-diketoandrost-1,4-diene compounds but only maintenance of that state by ADT with the added 6-methylene-3-keto-17-hydroxylandrost-1,4-diene compound so that effective binding of the hypothalamic receptor occurs to interfere with the downregulation of testosterone at the hypothalamus.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition comprising a sufficient amount of at least two ketosteroid derivatives of testosterone's metabolism in association with a pharmaceutically acceptable carrier for increasing testosterone's physiological levels in a male subject.

2. The composition according to claim 1, wherein said increase in testosterone's levels increases libido, muscle strength and/or athletic performance.

3. The composition according to claim 1, wherein said composition comprises four ketosteroid derivatives of testosterone's metabolism.

4. The composition according to claim 1, wherein said ketosteroid derivatives of testosterone are chosen from 3,17-diketoandrost-1,4,6-triene, 6-methylene-3-keto-17-hydroxylandrost-1,4-diene, 6-bromo-alpha-3,17-diketoandrost-1,4-diene and 6-bromo-beta-3,17-diketoandrost-1,4-diene.

5. The composition according to claim 3, wherein said four ketosteroid derivatives of testosterone's metabolism are 3,17-diketoandrost-1,4,6-triene, 6-methylene-3-keto-17-hydroxylandrost-1,4-diene, 6-bromo-alpha-3,17-diketoandrost-1,4-diene and 6-bromo-beta-3,17-diketoandrost-1,4-diene.

6. The composition according to claim 1, wherein said pharmaceutically acceptable carrier is a liposomal carrier bound to a saliva-absorbing carrier.

7. The composition according to claim 6, wherein said saliva-absorbing carrier is microcrystalline cellulose.

8. A method for improving a male's libido, muscle strength and/or athletic performances, which comprises administering a sufficient amount of the composition of claim 1.

9. The method according to claim 8, wherein said administration is peroral.

10. The method according to claim 8, wherein said administration is selected from the group consisting of transdermal, intranasal, and sublingual.

11. The method according to claim 8, wherein said amount is a daily total dosage of about 25 to 1000 mg.

12. The method according to claim 11, wherein said daily total dosage is divided at least in two equal dosages to be administered at twelve hours intervals.

13. The method according to claim 8, wherein said testosterone's levels are increased to supraphysiological levels.

14. The composition according to claim 8, wherein muscle size is increased.
